(19) United States
(12) Patent Application Publication
(10) Pub. No.: US 2024/0041404 A1
(43) Pub. Date: Feb. 8, 2024

(54) BIORESPONSIVE INTERFACES FOR THE ORAL CAVITY

(71) Applicants: President and Fellows of Harvard College, Cambridge, MA (US); Trustees of Tufts College, Medford, MA (US)

(72) Inventors: Fiorenzo G. Omenetto, Lexington, MA (US); Giusy Giusy, Medford, MA (US); Gili Naveh, Brookline, MA (US)

(21) Appl. No.: 18/348,717

(22) Filed: Jul. 7, 2023

Related U.S. Application Data
(63) Continuation of application No. PCT/US2022/070136, filed on Jan. 11, 2022.
(60) Provisional application No. 63/136,196, filed on Jan. 11, 2021.

(51) Int. Cl. A61B 5/00; A61B 5/145; A61B 10/00; A61B 5/1468

(52) U.S. Cl. CPC A61B 5/682; A61B 5/14539; A61B 5/14507; A61B 10/0051; A61B 5/1468

(57) ABSTRACT

The present disclosure provides an oral sampling device for chemical examination of an oral cavity. The oral sampling device includes an oral sampling support substrate, and a bioresponsive interface coupled to the oral sampling support substrate. The bioresponsive interface is composed of a biopolymer matrix comprising a sensing agent. In some aspects, the bioresponsive interface undergoes a color change in response to an environmental parameter (e.g., pH value) in the region of interest in the oral cavity.

### CROSS-REFERENCE TO RELATED APPLICATIONS

This application is a bypass continuation of International Application Serial No. PCT/US2022/070136, filed Jan. 11, 2022 (2095.0443), Int'l Publication No. 2022/174203. International Application Serial No. PCT/US2022/070136 is related to, claims priority to, and incorporates herein by reference for all purposes U.S. Provisional Patent Application No. 63/136,196, filed Jan. 11, 2021 (2095.0442). Each of the foregoing patent applications is incorporated herein by reference in their entirety for all purposes.

### STATEMENT REGARDING FEDERALLY SPONSORED RESEARCH

This invention was made with government support under grant W911QY-15-2-0001 awarded by the United States Army. The government has certain rights in the invention.

### BACKGROUND

The oral cavity provides an exceptional diagnostic environment. It is readily accessible with minimal to no invasiveness, offering access to blood as well as to mucosal samples from the tongue, cheeks and pharynx. Moreover, the oral cavity allows the collection of saliva with proficiency and minimal efforts. The non-invasive nature of saliva detection in real-time makes oral biomarker evaluation a potentially inexpensive and easy to use diagnostic approach. In the clinical practice, saliva evaluation currently allows one-time measurements thus preventing continuous multi-analyte tracking and, in many instances, causing diagnostically indicative fluctuations in local parameters to be missed. Despite the great potential, salivary diagnostics for domestic use has not yet transitioned into commercially available devices. The main limitations in salivary diagnostics are dictated by the lack of reproducible sampling techniques, analytical computation of low concentrations (i.e., range of pg-ng per μL) and dynamic levels of biomarkers intra- and inter-individuals.

Moreover, biomarkers levels and saliva composition vary throughout the day (e.g., following hormonal oscillations) making it an excellent tool for personalized diagnostics on one hand, however establishing the diagnostic correlations between analyte variations and specific diseases limited on the other.

Saliva is a hypotonic exocrine fluid consisting of 99% water and is the initiator of the digestion process. It mainly contains electrolytes, proteins, immunoglobulins, viral and bacterial genetic codes, as well as antimicrobial regulators and lubricating agents. Saliva is also an ion reservoir for oral pH regulation and enamel remineralization (i.e., maintaining neutral pH levels within the range 6.6-7.1). pH fluctuations can compromise oral health and specifically the tooth structure leading to the most prevalent oral disease: dental caries. Salivary pH below 6.6 is indicative of increased risk for dental caries, and it has been detected in cancer patients or people affected by Gastroesophageal Reflux (GERD). Further importance in detection and prevention comes from localized monitoring, which targets increase in acidic levels (i.e., below pH 5.5) between and around teeth indicating either the onset or presence of caries. Current detection methods such as clinical examination and x-rays can identify established carious lesions and are only available in dental practices.

### SUMMARY OF THE DISCLOSURE

The present disclosure addresses the aforementioned shortcomings by providing oral sampling devices that are configured to controllably access specific areas of the oral cavity and dynamically monitor environmental parameters (e.g., pH fluctuations). Such devices may be used outside of dental offices, and do not require x-rays to identify and monitor carious lesions in difficult to reach regions in the oral cavity.

An oral sampling device, as described herein, may have various configurations. The oral sampling device may include an oral sampling support substrate and a bioresponsive interface coupled to the oral sampling support substrate.

In one aspect, the oral sampling device is provided for chemical examination of an oral cavity. The oral sampling device includes an oral sampling support substrate, and a bioresponsive interface coupled to the oral sampling support substrate. The bioresponsive interface is composed of a biopolymer matrix comprising a sensing agent. In some aspects, the bioresponsive interface undergoes a color change in response to an environmental parameter (e.g., pH value) in the region of interest in the oral cavity.

These and other advantages and features of the present invention will become more apparent from the following detailed description of the preferred aspects of the present invention when viewed in conjunction with the accompanying drawings.

### DESCRIPTION OF THE DRAWINGS

The patent or application file contains at least one drawing executed in color. Copies of this patent or patent application publication with color drawing(s) will be provided by the Office upon request and payment of the necessary fee. The colorimetric nature of this invention required that certain color images be included with this filing, despite the nature of the drawings needing to be black and white. If the color version of any drawing is needed for patent examination persons, the inventors will provide any necessary color versions of the data to a patent examiner. If a reader desires the color version of the figures, please refer to a corresponding peer-reviewed journal publication describing the experiments carried forth in the examples section.

FIGS. 1(A-B) are schematic illustrations of an oral sampling device in accordance with some embodiments of the present disclosure.

FIGS. 2(A-B) are schematic illustrations of an oral sampling device in accordance with some embodiments of the present disclosure.

FIGS. 3(A-B) are schematic illustrations of an oral sampling device in accordance with some embodiments of the present disclosure.

FIGS. 4(A-B) are schematic illustrations of an oral sampling device in accordance with some embodiments of the present disclosure.

FIG. 5(A-B) are schematic illustrations of an edible matrix in accordance with some embodiments of the present disclosure.

FIG. 6 is a schematic illustration of a system configured to monitor and/or analyze a bioresponsive interface in accordance to embodiments of the present disclosure.

FIG. 7 is a schematic of biomaterial-based sensing mixes comprising silk fibroin and pH sensing molecules (i.e., commercially available pH indicators or naturally extracted from fruits and vegetables). The schematic shows the steps involved in the making of sensing interfaces that can be used to monitor pH variations within the oral cavity. Sensing mixes contain silk fibroin and a pH sensing molecule. The sensing mix is used to realize different types or intraoral sensing devices: (i) spray coated to generate pH detecting dental floss; (ii) dip coated highly absorbent paper points to detect pH in between teeth or inside teeth during root canal treatment; (iii) casted into molds to realize color changing candies able to sense pH variations.

FIG. 8 illustrates plots and images that characterize biomaterial-based sensing mixes used to spray coat dental floss and to dip coat highly absorbent paper points able to colorimetrically detect pH variations within the oral cavity. The plots and pictures show the color changes of indicators at pH values ranging between 3-8.5. (a) Commercial pH indicator nitrazine yellow (NY) embedded into a silk-based mix spray coated on dental floss: sensing range pH 4.5-7.5. (b) Mix of commercial pH indicators bromocresol green (BG)/chlorophenol red (CPR) spray coated on dental floss. The picture shows the dental floss exposed to different pH variations in 3 highlighted sections: enlargements at pH 4, 6, and 7.5 (i.e., from left to right). The plot shows that the sensing range falls within the pH interval 3-7. (c) Mix of commercial pH indicators, i.e., BG/CPR, embedded into a silk-based mix: sensing range pH 3-7. (d) Highly absorbent paper points dip coated with commercial pH indicators embedded into silk-based mixes. Left: BG/CPR point exposed to pH 5. Middle: NY color changing points exposed to pH 4, 5, 6, 7, and 8.5 (i.e., the arrow indicates the direction of paper points exposed to increased pH). Right: enlargement of point at pH 6. (e) Commercial pH indicator NY embedded into a silk-based mix: sensing range pH 4-7.5. The plots allow quantifying the signal as variations in either Red (i.e., CPR/BG, and NY for dental floss) or Euclidean Distance (ED) (i.e., CPR/BG, and NY for paper points) channel intensity vs pH.

FIG. 9 illustrates plots and images that characterize biomaterial-based sensing mixes used to dip coat highly absorbent paper points able to colorimetrically detect pH variations within the oral cavity. The plots and pictures show the color changes at pH values ranging between 3-8.5. (a) Anthocyanin extracted from blueberries (BB) embedded into a silk-based mix: sensing range pH 3.5-5, pH 6-7, and pH 8-8.5. (b) Highly absorbent paper points dip coated with anthocyanin extracted from blueberries (BB) embedded into silk-based mixes. Left: BB color changing points exposed to different pH 4, 5, 6, 7, and 8 (i.e., the arrow indicates the direction of paper points exposed to increased pH). Right: BB point exposed to pH 6. (c) Anthocyanin extracted from BB/RC (i.e., combined in ratio 1:2) embedded into a silk-based mix: sensing range pH 3-4, pH 4.5-5, pH 5.5-6, pH 6.5-7, and pH 7.5-8.5. (d) Highly absorbent paper points dip coated with anthocyanin extracted from a combination of BB and red cabbage (RC) embedded into silk-based mixes. Left: RC/BB point exposed to pH 6. Middle: RC/BB color changing points exposed to different pH 4, 5, 6, 7, and 8 (i.e., the arrow indicates the direction of paper points exposed to increased pH). The plots allow quantifying the signal as variations in either Red (i.e., BB paper points) or Green (i.e., BB/RC paper points) channel intensity vs pH.

FIG. 10 illustrates plots and images that characterize mouth conformable interfaces based on biomaterial-based sensing mixes able to colorimetrically detect pH variations within the oral cavity. The plots and pictures show the color changes at pH values ranging between 3-8.5. (a) Examples of colorimetric mouth conformable sensing interfaces that can monitor pH variations within the oral cavity. They can be realized in the format of spray coated dental aligners and edible lollipops able to evaluate the buffering capacity of saliva inside the mouth. The inset shows a dental aligner exposed to pH 3 (i.e., acid) and pH 8.5 (i.e., base). (b) Lollipop based on silk fibroin embedding anthocyanins extracted from BB. The lollipop is exposed to pH variations in 4 highlighted sections: pH 4, 6, 7, and 8.5 (i.e., arrows point at the direction of increased pH). The enlargement shows the color changes recorded at pH 8.5 (i.e., top) and pH 4 (i.e., bottom). (c) pH indicator extracted from nectarine skins (N) embedded into a silk mix molded&casted in the format of a lollipop: sensing range pH 3-4 and pH 7-8. (d) pH indicator extracted from blueberries (BB) embedded into a silk mix molded and casted in the format of a lollipop: sensing range pH 4.5-7. The plots allow quantifying the signal as variations in either Red (i.e., N lollipops) or Euclidean Distance (ED) (i.e., BB lollipops) channel intensity vs pH.

FIG. 11 is a characterization of NY based pH sensing mix via UV-VIS absorbance measurements. The pictures show the color changes of NY silk fibroin mix at pH values ranging between 3-8.5. Absorbance peaks are recorded within the range 350-800 nm: arrows (i.e., in correspondence of two peaks at 465 nm and 590 nm) point at spectra recorded at decreasing or increasing pH values. Intensity variations (i.e., recorded at 465 nm and 590 nm) are plotted against the pH recorded with a standard pH meter of each color changing solution.

FIG. 12 is a characterization of CPR/BG (i.e., ratio 1:1) based pH sensing mix via UV-VIS absorbance measurements. The pictures show the color changes of CPR/BG silk fibroin mix at pH values ranging between 3-8.5. Absorbance peaks are recorded within the range 350-800 nm: arrows (i.e., in correspondence of three peaks at 435 nm, 575 nm, and 630 nm) point at spectra recorded at decreasing or increasing pH values. Intensity variations (i.e., recorded at 435 nm, 575 nm, and 630 nm) are plotted against the pH recorded with a standard pH meter of each color changing solution.

FIG. 13 is a characterization of BB based pH sensing mix via UV-VIS absorbance measurements. The pictures show the color changes of BB silk fibroin mix at pH values ranging between 3-8. Absorbance peaks are recorded within the range 400-800 nm: arrows (i.e., in correspondence of two peaks at 525 nm and 610 nm) point at spectra recorded at decreasing or increasing pH values. Intensity variations (i.e., recorded at 525 nm and 610 nm) are plotted against the pH recorded with a standard pH meter of each color changing solution.

FIG. 14 is characterization of BB/RC (i.e., ratio 1:2) based pH sensing mix via UV-VIS absorbance measurements. The pictures show the color changes of RC/BB silk fibroin mix at pH values ranging between 3-8. Absorbance peaks are recorded within the range 400-800 nm: arrows (i.e., in correspondence of two peaks at 530 nm and 610 nm) point at spectra recorded at decreasing or increasing pH values. Intensity variations (i.e., recorded at 530 nm and 610 nm) are plotted against the pH recorded with a standard pH meter of each color changing solution.

FIG. 15 is characterization of RC-based and biomaterial-based sensing mixtures. (a) Characterization of RC based pH sensing mix via UV-VIS absorbance measurements. The pictures show the color changes of RC silk fibroin mix at pH values ranging between 3-8. Absorbance peaks are recorded within the range 400-800 nm: arrows (i.e., in correspondence of two peaks at 530 nm and 605 nm) point at spectra recorded at decreasing or increasing pH values. Intensity variations (i.e., recorded at 530 nm and 605 nm) are plotted against the pH recorded with a standard pH meter of each color changing solution. (b) Characterization of biomaterial-based sensing mixture used to dip coat highly absorbent paper points able to colorimetrically detect pH variations within the oral cavity. The pictures show the color changes of RC indicator at pH values ranging between 3-8, with sensing ranges of pH 3-3.5, pH 5-7, and pH 7.5-8. The plot allows quantifying the signal as variation in the Green channel intensity vs pH.

FIG. 16 is characterization of BB/RC-based and biomaterial-based sensing mixtures. (a) Characterization BB/RC (i.e., ratio 2:1) based pH sensing mix via UV-VIS absorbance measurements. The pictures show the color changes of RC/BB silk fibroin mix at pH values ranging between 3-8. Absorbance peaks are recorded within the range 400-800 nm: arrows (i.e., in correspondence of two peaks at 525 nm and 610 nm) point at spectra recorded at decreasing or increasing pH values. Intensity variations (i.e., recorded at 525 nm and 610 nm) are plotted against the pH recorded with a standard pH meter of each color changing solution. (b) Characterization of biomaterial-based sensing mixture used to dip coat highly absorbent paper points able to colorimetrically detect pH variations within the oral cavity. The pictures show the color changes of RC/BB indicators at pH values ranging between 3-8, with sensing ranges of pH 3-4 and pH 5.5-7. The plot allows quantifying the signal as variation in the Red channel intensity vs pH.

FIG. 17 is characterization of nectarine skin-based and biomaterial-based sensing mixtures. (a) Characterization of nectarine skins (i.e., N) based pH sensing mix via UV-VIS absorbance measurements. The pictures show the color changes of N silk fibroin mix at pH values ranging between 3-8. Absorbance peaks are recorded within the range 400-800 nm: arrows (i.e., in correspondence of two peaks at 515 nm and 600 nm) point at spectra recorded at decreasing or increasing pH values. Intensity variations (i.e., recorded at 515 nm and 600 nm) are plotted against the pH recorded with a standard pH meter of each color changing solution. (b) Characterization of biomaterial-based sensing mixture used to dip coat highly absorbent paper points able to colorimetrically detect pH variations within the oral cavity. The pictures show the color changes of N indicator at pH values ranging between 3-8.5, with sensing ranges of pH 3.5-5.5, and pH 7-7.5. The plot allows quantifying the signal as variation in the Green channel intensity vs pH.

FIG. 18 is characterization of N/BB-based and biomaterial-based sensing mixtures. (a) Characterization of N/BB (i.e., ratio 2:1) based pH sensing mix via UV-VIS absorbance measurements. The pictures show the color changes of N/BB silk fibroin mix at pH values ranging between 3-8. Absorbance peaks are recorded within the range 400-800 nm: arrows (i.e., in correspondence of two peaks at 520 nm and 625 nm) point at spectra recorded at decreasing or increasing pH values. Intensity variations (i.e., recorded at 520 nm and 625 nm) are plotted against the pH recorded with a standard pH meter of each color changing solution. (b) Characterization of biomaterial-based sensing mixture used to dip coat highly absorbent paper points able to colorimetrically detect pH variations within the oral cavity. The pictures show the color changes of N/BB (i.e., ratio 2:1) indicators at pH values ranging between 3-8, with sensing ranges of pH 4-6.5 and pH 6.5-7. The plot allows quantifying the signal as variation in the Green channel intensity vs pH.

### DETAILED DESCRIPTION

Before the present invention is described in further detail, it is to be understood that the invention is not limited to the particular embodiments described. It is also to be understood that the terminology used herein is for the purpose of describing particular embodiments only, and is not intended to be limiting. The scope of the present invention will be limited only by the claims. As used herein, the singular forms “a”, “an”, and “the” include plural embodiments unless the context clearly dictates otherwise.

It should be apparent to those skilled in the art that many additional modifications beside those already described are possible without departing from the inventive concepts. In interpreting this disclosure, all terms should be interpreted in the broadest possible manner consistent with the context. Variations of the term “comprising”, “including”, or “having” should be interpreted as referring to elements, components, or steps in a non-exclusive manner, so the referenced elements, components, or steps may be combined with other elements, components, or steps that are not expressly referenced. Embodiments referenced as “comprising”, “including”, or “having” certain elements are also contemplated as “consisting essentially of” and “consisting of” those elements, unless the context clearly dictates otherwise. It should be appreciated that aspects of the disclosure that are described with respect to a system are applicable to the methods, and vice versa, unless the context explicitly dictates otherwise.

Numeric ranges disclosed herein are inclusive of their endpoints. For example, a numeric range of between 1 and 10 includes the values 1 and 10. When a series of numeric ranges are disclosed for a given value, the present disclosure expressly contemplates ranges including all combinations of the upper and lower bounds of those ranges. For example, a numeric range of between 1 and 10 or between 2 and 9 is intended to include the numeric ranges of between 1 and 9 and between 2 and 10.

Oral health monitoring is highly desired, especially for in home use, however current methods are not sensitive enough and technically convoluted for this purpose. The present disclosure provides an approach of incorporating sensing agents or bioactive materials into oral sampling support substrates (i.e., oral appliances) to transform them into bioresponsive interfaces. Edible sampling devices having bioresponsive interfaces were also developed. The edible sampling devices may have a form factor of candy that dynamically responds to environmental parameters in the oral cavity (e.g., pH changes in saliva and/or pH of oral surfaces). Such devices and interfaces allow for early detection medical conditions, such as caries, providing low-cost point of care devices that respond in real-time by detecting chemical parameters in biological fluids, thus bringing monitoring to home settings instead of clinical practices.

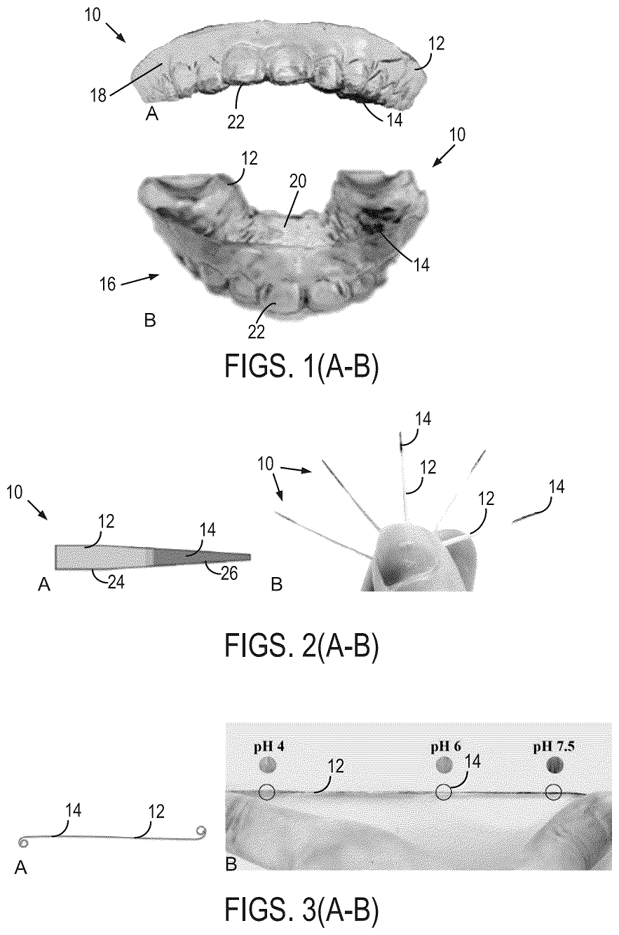

Referring to FIGS. 1(A-B), an oral sampling device 10 is illustrated in accordance with some aspects of the present disclosure. The oral sampling device 10 includes an oral sampling support substrate 12 and a bioresponsive interface 14 coupled to the oral sampling support substrate 12.

The oral sampling device 10 and/or the bioresponsive interface 14 can change color upon contacting a tissue or tissue surrounding environment in response to local ion or chemical molecular concentration. The change in color can report chemical information regarding the local environment for tissues and/or organs. In some cases, the chemical information can be related to teeth, gums, pharynx, trachea, esophagus, gastral tract, lungs, or a combination thereof.

In some aspects, the term “oral sampling support substrate” may refer to a dental or orthodontic tool sized to fit in a subject's mouth and sample a region of interest in the oral cavity. In some aspects, the oral sampling support substrate 12 is sized to be received within the oral cavity, and/or sized to fit between a subject's teeth, and/or is configured to wrap around at least a portion of a subject's tooth. Exemplary oral sampling support substrates 12 include, but are not limited to, dental floss, a dental pick (e.g., toothpick), a paper point, toothbrush, rubber tip stimulator (e.g., gum stimulator), tongue cleaner, mouth tray (e.g., mouth guard, grind guard), dental aligner, retainer, tweezer, dental mirror, dental scaler, tarter scraper, test strip, dry or wet swabs (e.g., cotton swab, rayon tipped swabs, polyester tipped swabs, foam tipped swabs, flocked swabs), stem or stick (e.g., stem for a lollipop), and combinations thereof.

In some aspects, the bioresponsive interface 14 includes a biopolymeric matrix. As used herein, the term “biopolymeric matrix” may refer to a biologically compatible polymer matrix or biopolymer matrix material. In some aspects, the biopolymer comprises a fragment or variant of a biological polymer. Exemplary biologically compatible polymers or biopolymers include, but are not limited to, fibroins, silk fibroin, actins, collagens, catenins, claudins, coilins, elastins, elaunins, extensins, fibrillins, keratins, lamins, laminins, fibrions, tublins, viral structural proteins, zein proteins (seed storage protein), polyethylene oxide (PEO), polyethylene glycol (PEG), collagen, fibronectin, keratin, polyaspartic acid, polylysine, alginate, chitosan, chitin, hyaluronic acid, pectin, polycaprolactone, polylactic acid, polyglycolic acid, polyhydroxyalkanoates, dextrans, polyanhydrides and any combinations thereof.

In some aspects, the biopolymer comprises silk fibroin. Silk fibroin is derived from Bombyx mori silkworm cocoons, is a biocompatible and biodegradable material that degrades slowly in the body, is readily modified into a variety of formats, and generates mechanically robust materials.

As used herein, the term “silk fibroin” refers to silk fibroin protein whether produced by silkworm, spider, or other insect, or otherwise generated (Lucas et al., Adv. Protein Chem., 13: 107-242 (1958)). Any type of silk fibroin can be used in different aspects described herein. Silk is naturally produced by various species, including, without limitation: Antheraea mylitta; Antheraea pernyi; Antheraea yamamai; Galleria mellonella; Bombyx mori; Bombyx mandarina; Galleria mellonella; Nephila clavipes; Nephila senegalensis; Gasteracantha mammosa; Argiope aurantia; Araneus diadematus; Latrodectus geometricus; Araneus bicentenarius; Tetragnatha versicolor; Araneus ventricosus; Dolomedes tenebrosus; Euagrus chisoseus; Plectreurys tristis; Argiope trifasciata; and Nephila madagascariensis. Silk fibroin produced by silkworms, such as Bombyx mori, is the most common and represents an earth-friendly, renewable resource. For instance, silk fibroin used in a silk film may be attained by extracting sericin from the cocoons of B. mori. Organic silkworm cocoons are also commercially available. There are many different silks, however, including spider silk (e.g., obtained from Nephila clavipes), transgenic silks, genetically engineered silks, such as silks from bacteria, yeast, mammalian cells, transgenic animals, or transgenic plants, and variants thereof, that can be used. See, e.g., WO 97/08315 and U.S. Pat. No. 5,245,012, each of which is incorporated herein as reference in its entirety.

In some aspects, silk fibroin scaffolds comprising silk fibroin may be made using one or more silk solutions, which are known to be highly customizable and allow for the production of any of a variety of end products. As such, in some aspects, scaffold matrix materials may be produced using any of a variety of silk solutions. Preparation of silk fibroin solutions has been described previously, e.g., in WO 2007/016524, which is incorporated herein by reference in its entirety.

In accordance with various aspects, a silk solution may comprise any of a variety of concentrations of silk fibroin. In some aspects, a silk solution may comprise 0.1 to 30% by weight silk fibroin. In some aspects, a silk solution may comprise between about 0.5% and 30% (e.g., 0.5% to 25%, 0.5% to 20%, 0.5% to 15%, 0.5% to 10%, 0.5% to 5%, 0.5%) to 1.0%) by weight silk fibroin, inclusive. In some aspects, a silk solution may comprise at least 0.1% (e.g., at least 0.5%, 1%, 2%, 3%, 4%, 5%, 6%, 7%, 8%, 9%, 10%, 15%, 20%), 25%)) by weight silk fibroin. In some aspects, a silk solution may comprise at most 30% (e.g., at most 25%, 20%, 15%, 14%, 13%, 12% 11%, 10%, 5%, 4%, 3%, 2%,1%) by weight silk fibroin.

In accordance with various aspects, silk used in the provided device is degummed silk (i.e. silk fibroin with at least a portion of the native sericin removed). Degummed silk can be prepared by any conventional method known to one skilled in the art. For example, B. mori cocoons are boiled for a period of pre-determined time in an aqueous solution. Generally, longer degumming time generates lower molecular silk fibroin. In some aspects, the silk cocoons are boiled for 60 minutes to 120 minutes, or longer. Additionally or alternatively, in some aspects, silk cocoons can be heated or boiled at an elevated temperature. For example, in some aspects, silk cocoons can be heated or boiled at 100° C. to about 120.0° C., or higher.

In some aspects, such elevated temperature can be achieved by carrying out at least portion of the heating process (e.g., boiling process) under pressure. For example, suitable pressure under which silk fibroin fragments described herein can be produced are typically between about 10-40 psi.

In some aspects, the aqueous solution used in the process of degumming silk cocoons comprises about 0.02M Na₂CO₃. The cocoons are rinsed, for example, with water to extract the sericin proteins. The degummed silk can be dried and used for preparing silk powder. Alternatively, the extracted silk can dissolved in an aqueous salt solution. Salts useful for this purpose include lithium bromide, lithium thiocyanate, calcium nitrate or other chemicals capable of solubilizing silk. In some aspects, the extracted silk can be dissolved in about 8M-12 M LiBr solution. The salt is consequently removed using, for example, dialysis.

In some aspects, the silk fibroin is substantially depleted of its native sericin content (e.g., 5% (w/w) or less residual sericin in the final extracted silk). In some aspects, the silk fibroin is entirely free of its native sericin content. As used herein, the term “entirely free” (i.e. “consisting of” terminology) means that within the detection range of the instrument or process being used, the substance cannot be detected or its presence cannot be confirmed. In some aspects, the silk fibroin is essentially free of its native sericin content. As used herein, the term “essentially free” (or “consisting essentially of”) means that only trace amounts of the substance can be detected, is present in an amount that is below detection, or is absent.

If necessary, the silk solution can be concentrated using, for example, dialysis against a hygroscopic polymer, for example, PEG, a polyethylene oxide, amylose or sericin. In some aspects, the PEG is of a molecular weight of 8,000-10,000 g/mol and has a concentration of about 10% to about 50% (w/v). A slide-a-lyzer dialysis cassette (Pierce, MW CO 3500) can be used. However, any dialysis system can be used. The dialysis can be performed for a time period sufficient to result in a final concentration of aqueous silk solution between about 10% to about 30%. In most cases dialysis for 2-12 hours can be sufficient. See, for example, International Patent Application Publication No. WO 2005/012606, the content of which is incorporated herein by reference in its entirety. Another method to generate a concentrated silk solution comprises drying a dilute silk solution (e.g., through evaporation or lyophilization). The dilute solution can be dried partially to reduce the volume thereby increasing the silk concentration. The dilute solution can be dried completely and then dissolving the dried silk fibroin in a smaller volume of solvent compared to that of the dilute silk solution.

In some aspects, a silk fibroin solution can optionally, at a suitable point, be filtered and/or centrifuged. For example, in some aspects, a silk fibroin solution can optionally be filtered and/or centrifuged following the heating or boiling step. In some aspects, a silk fibroin solution can optionally be filtered and/or centrifuged following the dialysis step. In some aspects, a silk fibroin solution can optionally be filtered and/or centrifuged following the step of adjusting concentrations. In some aspects, a silk fibroin solution can optionally be filtered and/or centrifuged following the step of reconstitution. In any of such aspects, the filtration and/or centrifugation step(s) can be carried out to remove insoluble materials. In any of such aspects, the filtration and/or centrifugation step(s) can be carried out to selectively enrich silk fibroin fragments of certain molecular weight(s).

In some aspects, the biopolymer or biocompatible polymer solution is coated onto at least a portion of the oral sampling support substrate 12. The coating may be applied through any suitable method, including dip coating or spray coating.

Referring back to FIGS. 1(A-B), the bioresponsive interface 14 may include a sensing agent. As used herein, the term “sensing agent” may refer to a compound or chemical moiety that alters the bioresponsive interface 14 from a first chemical-physical state to a second chemical-physical state that is indicative of one or more parameter in the oral cavity. For example, the bioresponsive interface 14 may change color in response to a change in pH or a concentration of a chemical species (e.g., biomarker, polypeptide, contaminant, or toxin) in the oral cavity.

In some aspects, the sensing agent comprises a pH sensing agent. Exemplary pH sensitive agents include compounds or chemical moieties comprising, but are not limited to, bromocresol green, chlorophenol red, nitrazine yellow, and combinations thereof. In some aspects, the pH sensitive compound or chemical moiety is derived or extracted from a natural source, such as fruits (e.g., blueberries, nectarine skins) and vegetables (e.g., red cabbage).

Exemplary pH sensitive compounds or chemical moieties derived from a natural source include anthocyanins (e.g., extracted from Blueberries or commercially available powders featuring anthocyanins extracted from red cabbage, red radish, etc.) and carotenoids (e.g., extracted from nectarine skins, tomatoes, etc.).

In some aspects, the pH sensitive agent is present in the bioresponsive interface 14 at a concentration sufficient to alter the color of the bioresponsive interface 14 in the presence of a change in an environmental parameter. In some aspects, the one or more pH sensing agent is present in the bioresponsive interface 14 at a concentration that falls in the range from 5% to 50% w/v, based on the total volume of the bioresponsive interface. In some embodiments, the concentration of the pH sensing agent is at least 5% w/v, or at least 10% w/v, or at least 15% w/v, or at least 20% w/v, or at least 25% w/v, or at least 30% w/v, or at least 35% w/v, to less than 40% w/v, or less than 45% w/v, or less than 50% w/v.

In some non-limiting examples, the one or more pH sensing agent is present in the bioresponsive interface 14 at a ratio of pH sensing agent to water from 5:1 to 1:5. In some embodiments, the one or more pH sensing agent is present in a ratio of pH sensing agent to water of at least 5:1, at least 4:1, at least 3:1, at least 2:1, at least 1:1, at least 1:2, at least 1:3, to less than 1:4, less than 1:5, and values between the specified bounds.

The oral sampling device 10 may have various form factors. As shown by FIGS. 1(A-B), the oral sampling device 10 includes an oral sampling support substrate 12 in the form of a mouth tray or dental aligner. The mouth tray or dental aligner includes a U-shaped base 16 having an outer labial wall 18, an inner lingual wall 20, and an intervening tray floor 22. The intervening tray floor 22 of the dental aligner may include a positive mold of at least one of, or all of, the subject's teeth. Although not depicted in FIGS. 1(A-B), a mouth tray may have a flat or substantially flat intervening tray floor 22.

In some aspects, the bioresponsive interface 14 is coated on an exterior surface of the outer labial wall 18 (e.g., may be configured to contact lips of an oral cavity), an outer surface of the inner lingual wall 20 (e.g., may be configured to contact a tongue in the oral cavity), and/or an outer surface of the intervening tray floor 22 (e.g., may be configured to contact teeth in the oral cavity). In some aspects, the bioresponsive interface 14 is coated on an interior surface of the outer labial wall 18 (e.g., may contact a facial and/or buccal surface of a subject's teeth), an interior surface of the inner lingual wall 20 (e.g., may contact a lingual surface of a subject's teeth), and/or an interior surface of the intervening tray floor 22 (e.g., may contact an occlusal surface of a subject's teeth). Alternatively or additionally, the bioresponsive interface may be intermixed within a body of the outer labial wall 18, the inner lingual wall 20, and/or the intervening tray floor 22. For example, the mouth tray or dental aligner may be formed from a biopolymeric matrix described herein, and the sensing agent may be mixed therein.

Referring to FIGS. 2(A-B), in some aspects, the oral sampling device 10 has a form factor of a paper point. The paper point may have a cylindrical body 24 and at least one tapered end 26. The paper point may be composed of an absorbent material. Suitable absorbent materials include, but are not limited to, cellulose fibers, hemicellulose fibers, polyvinyl material, polyester material, hydrocolloid material, and combinations thereof. In some aspects, the length of the absorbent insert is from 5 mm to 30 mm, the diameter of the cylindrical body may range from 1 mm to 5 mm, and the diameter of the tapered end may be less than 3 mm, or less than 2 mm, or less than 1 mm, or less than 0.5 mm, or less than 0.01 mm. The taper may range from 0 to 10 degrees. In some aspects, the paper point includes a taper that extends an entire length of the paper point.

In some aspects, the bioresponsive interface 14 may coat at least a portion of the tapered end 26 of the paper point. In some aspects, the bioresponsive interface 14 coats the cylindrical body 24. In some aspects, the bioresponsive interface 14 coats both the cylindrical body 24 and the tapered end 26. In some aspects, a tip of the tapered end remains uncoated. An uncoated region on the tapered end may facilitate absorption of fluid in the oral cavity into the paper point, and may also reduce leaching of the bioresponsive interface 14.

Referring to FIGS. 3(A-B), in some aspects, the oral sampling device 10 has a form factor of dental floss. The dental floss may be composed of nylon, polytetrafluoroethylene (PTFE or Teflon®), polypropylene, polyethylene, styrene butadyene copolymers, or combinations thereof. The dental floss may be composed of a monofilament or may be composed of a plurality of filaments (e.g., 2 to 300). In some embodiments, the floss has a denier from 100 to 1400.

In some aspects, the bioresponsive interface 14 may coat at least a portion of an exterior surface of the dental floss. In some aspects, the bioresponsive interface 14 coats the entire exterior surface of the dental floss.

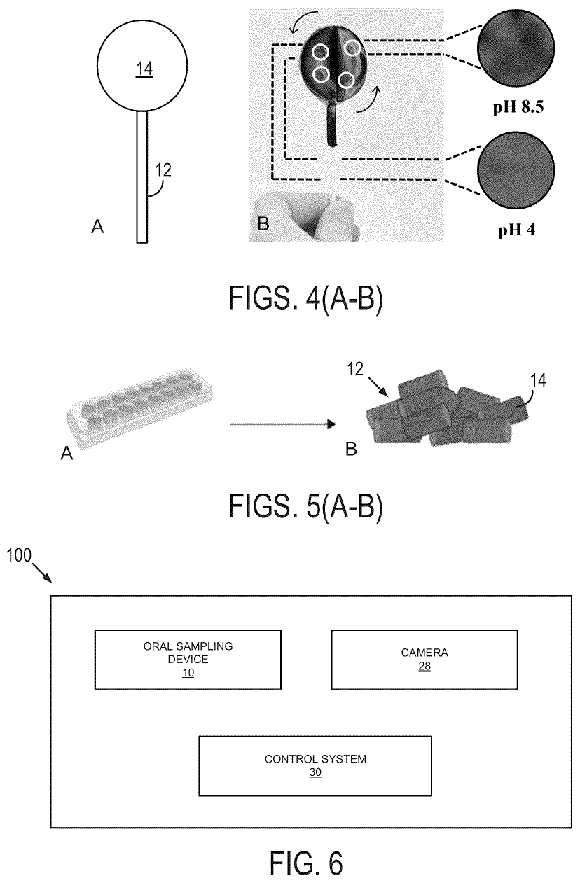

Referring to FIGS. 4(A-B), the oral sampling support substrate 12 may be in the form of a lollipop. The oral sampling support substrate 12 may be an elongate stick, and the bioresponsive interface 14 may be in the form of an edible matrix mounted on the elongate stick. The elongate stick may be composed of cellulosic materials (e.g., tightly-wrapped white or printed paper). Referring to FIGS. 5(A-B), in some aspects, the oral sampling device is composed of an edible matrix (e.g., candy) composed of a biopolymeric matrix, the sensing agent, and one or more additive. The edible matrix may be free of an oral sampling support substrate 14.

Suitable additives for the edible matrix may include a candy base (e.g., sugar, sugar free vehicles), such as sucrose, maltose, lactose, dextrose, PEG 600 and 800, mannitol, sorbitol, lactose, calcium sulphate, calcium carbonate, dicalcium phosphate, microcrystalline cellulose, and combinations thereof. In some aspects, suitable additives for the edible matrix include binders including, but not limited to, acacia, corn syrup, sugar syrup, gelatin, polyvinyl pyrollidone, tragacanth and methylcellulose. In some aspects, suitable additives for the edible matrix include lubricants including, but not limited to, stearic acid, magnesium stearate, calcium stearate, polyethylene glycol, vegetable oils and fats.

In some aspects, suitable additives for the edible matrix include flavoring agents including, but not limited to, menthol, eucalyptus oil, cherry flavor, and spearmint. In some aspects, suitable additives for the edible matrix include coloring agents including, but not limited to, water soluble and lakolene dyes, food and cosmetic colors, coloring paste and cubes. In some aspects, suitable additives for the edible matrix include whipping agents including, but not limited to, milk protein (e.g., Casein), egg albumin, gelatin, xanthan gum, starch, pectin, algin, and carrageenan. In some aspects, suitable additives for the edible matrix include humectants including, but not limited to, glycerin, propylene glycol, and sorbitol.

In some aspects, suitable additives for the bioresponsive interface 14 can include one or more stabilizers. Examples of suitable stabilizers include, but are not limited to, gelatin, cellulose, starch, alginate, or a combination thereof.

In some aspects, the present disclose provides a system for analyzing the bioresponsive interfaces 14. Referring to FIG. 6, the system 100 includes the oral sampling device such as those described herein, a camera 28 configured to photograph a region of interest on the oral sampling device 10, and a control system 30 having a memory containing a machine readable medium comprising machine executable code having stored instructions thereon for performing a method of analyzing the bioresponsive interfaces. The control system 30 is in electrical communication with the memory, and optionally the camera 28 and oral sampling device 10. The control system 30 is configured to execute the machine executable code to cause the control system 30 to determine a pH of the region of interest on the oral sampling device 10 based on a color in the region of interest. The control system 30 is further configured to output a report that includes the pH of the region of interest.

In some aspects, the report includes a map of pH values across the region of interest, a likelihood that the pH value in the region of interest is indicative of a medical condition (e.g., dental caries, Gastroesophageal reflux disease (GERD), or cancer. In some aspects, the report includes a remedy to increase or decrease the pH in the region of interest (i.e., instructions or recommendations to brush the subject's teeth, eat a certain food type to alter the pH, etc).

### Examples

The following examples will enable one of skill in the art to more readily understand the principles thereof. The following examples are presented by way of illustration and are not meant to be limiting in any way.

Materials:

Sodium carbonate, lithium bromide, bromocresol green sodium salt (BG), nitrazine yellow (NY), and chlorophenol red (CPR) were purchased from Sigma-Aldrich (USA). Ethanol (100%) was purchased from Fisher Scientific. All chemicals were used as received and they followed trace metal standard, when possible. Blueberries and nectarines were purchased in a local supermarket. Anthocyanins powders extracted from red cabbage were purchased from Latte Powder. Silk cocoons of Bombix Mori silkworm were purchased from Tajima Shoji (Japan). Deionized water with resistivity of 18.2 MΩ cm was obtained with a Milli-Q reagent-grade water system and used to prepare aqueous solutions. Dental floss from Reach and highly absorbent paper points from Dentsply Maillefer were employed as substrates for making mouth conformable colorimetric interfaces.

Silk Fibroin Solution Preparation:

Silk fibroin was prepared by boiling finely chopped Bombyx Mori silk cocoons in a solution of 0.02 M sodium carbonate to remove sericin for 30 minutes. Overnight-dried silk fibroin was added to 9.3 M LiBr solution and stored at 60° C. to dissolve fibers into aqueous solution. Pure silk solution (˜7-8%) was collected after dialysis (Fisherbrand, MWCO 3.5 KDa) for 48 hours.

Alternatively, silk fibroin was prepared by boiling finely chopped Bombyx Mori in a solution of 0.02 M sodium carbonate to remove the sericin layer for 30 minutes. The fibers were washed three times for 20 minutes in deionized water and dried overnight. They were dissolved in a solution of lithium bromide (i.e., 9.3 M) at 60° C. for 4 hours. A 20 wt % solution was obtained and dialyzed against deionized water for 2 days, changing the deionized water 6 times at regular intervals. The final solution was centrifuged twice at a speed of 9000 rpm, at 4° C., for 20 minutes and then filtered to obtain a 7-8 wt % silk fibroin solution.

Blueberry Cocktail Preparation:

Blueberries were weighed, rinsed with water, and crushed with a blender. The crushed mixture was transferred in a beaker and the blender was washed with deionized water (ratio water/fruit 1:1 w/w) then added to the blueberry mixture in the beaker. The blueberry cocktail was heated up to 85° C. and thickened for 40 minutes. The cocktail was cooled down and filtered 3 times until the final mixture was clear and ready to be used to functionalize mouth conformable interfaces.

Carotene Cocktail Preparation:

Nectarines were rinsed and peeled. Nectarine skins were weighed and packed in a beaker with a mix of deionized water and ethanol (i.e., ratio 2:1 v/v) double the weight of the starting material. Nectarine skins were kept in infusion overnight at room temperature. The skins were removed from the solution that was filtered to obtain a clear solution ready for use to functionalize and make mouth conformable interfaces.

Biomaterial-Based Cocktails for Spray Coating:

Biomaterial-based cocktails were realized mixing pure silk solution (i.e., final concentration of 8%) with commercially available pH indicators (i.e., NY 0.75 mg/mL; a combination of BG (0.5 mg/mL)/CPR (0.75 mg/mL)) and directly spray coated on dental floss.

Biomaterial-Based Cocktails for Dip Coating:

Biomaterial-based cocktails were realized mixing pure silk solution (i.e., final concentration of 5%) with commercially available pH indicators (i.e., NY, 0.75 mg/mL; BG, 0.5 mg/mL; CPR, 0.75 mg/mL)), anthocyanins from blueberries (i.e., ratio silk/blueberry cocktail 1:5 v/v), red cabbage (i.e., powder extract 10 mg/mL), and carotene extracted from nectarine skins (i.e., ratio silk/nectarine skin solutions 1:5 v/v)^{[24]}. The different cocktails used to dip coat highly absorbent paper points embedded one or a combination of pH indicators: i.e., BG/CPR, ratio 1:1 v/v; BB/RC, ratio 1:2 v/v; BB/RC, ratio 1:1 v/v; BB/RC, ratio 2:1 v/v; BB/N, ratio 1:2 v/v.

Biomaterial-Based Cocktails for Candies Making:

Pure silk solutions (i.e., initial concentration 14%) were mixed with anthocyanins extracted from blueberries (ratio silk/blueberries 1:1 v/v) or carotene extracted from nectarine skins (ratio silk/nectarines 1:2 v/v). The cocktails were transferred on round silicone molds to dry at room temperature and obtain edible color changing candies.

Responsive pH Sensing Cocktails Preparation and Functionalization of Dental Floss:

Biomaterial-based cocktails were realized mixing pure silk solution (i.e., final concentration of 8%) with pH indicators (i.e., NY 0.75 mg/mL; BG (0.5 mg/mL)/CPR (0.75 mg/mL)) and directly spray coated on dental floss.

Responsive pH Sensing Cocktails Preparation and Functionalization of Paper Points:

Biomaterial-based cocktails were realized mixing pure silk solution (i.e., final concentration of 5%) with commercially available pH indicators (i.e., NY 0.75 mg/mL; BG (0.5 mg/mL)/CPR (0.75 mg/mL)), anthocyanins extracted from blueberries (i.e., ratio silk/blueberries solutions 1:5 v/v) and red cabbage (i.e., powder extract 10 mg/mL), carotene extracted from nectarine skins (i.e., ratio silk/nectarine skin solutions 1:5 v/v). The different cocktails embed one or a combination of pH indicators (i.e., BG/CPR, BB/RC 1:2 v/v, BB/RC 1:1 v/v, BB/RC 2:1 v/v, BB/N 1:2 v/v) that were used to dip coat highly absorbent paper points.

Responsive pH Sensing Cocktails Preparation and Making of Candy-Like Devices:

Pure silk solutions (i.e., initial concentration 14%) were mixed with anthocyanins extracted from blueberries (ratio silk/blueberries 1:1 v/v) or carotene extracted from nectarine skins (ratio silk/nectarines 1:2 v/v). The mixes were transferred on molds to dry at room temperature and obtain edible color changing candies.

Analysis of Colorimetric pH Sensing Interfaces:

Biomaterial-based cocktails were first characterized in liquid format using the spectrophotometer Synergy HT from BioTex. Spectra were acquired in the range 400-800 nm, at steps of 5 nm. Mouth conformable interfaces in the form of colorimetric dental floss and highly absorbent paper points were analyzed collecting images using an electronic reader (i.e., 8-bit Laser Jet Pro MFP M127fn scanner from HP (USA), 24-bit color depth and resolution of 600 dpi), or a camera (i.e., Canon EOS Rebel T1i) in controlled lighting conditions. Mouth conformable interfaces in the form of colorimetric edible lollipops can be monitored collecting images using a photo camera. Color changing substrates were photographed with a Canon EOS Rebel T1i in controlled lighting conditions. ImageJ allowed quantifying the signal as variations in the Red or Green channel intensities or a combination of RGB channels intensities expressed in terms of Euclidean Distance (ED) (i.e., \sqrt{RED²+GREEN²+BLUE²}).

Performances of Colorimetric Cocktails

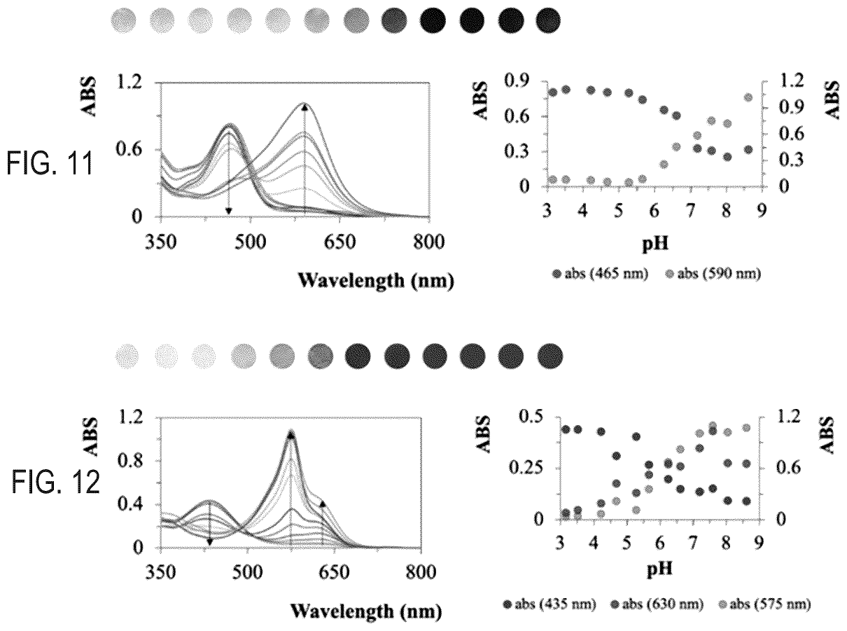

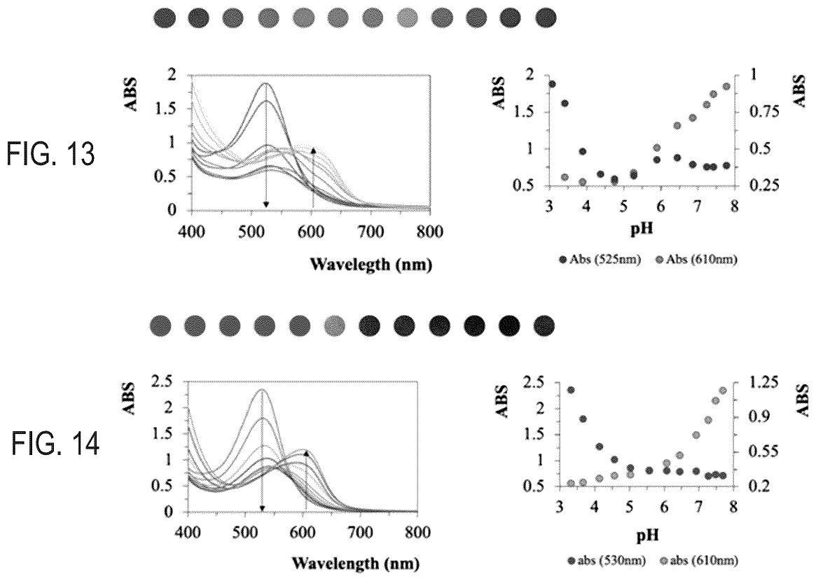

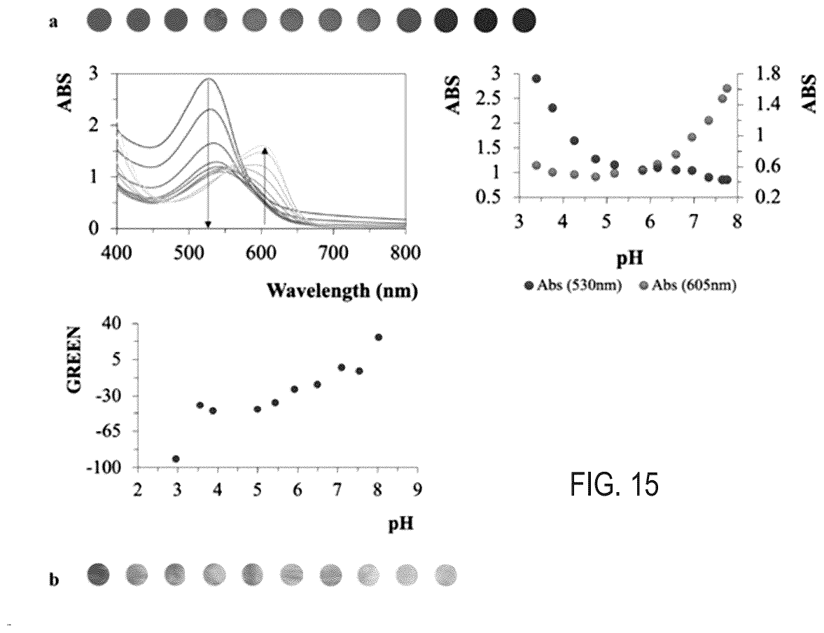

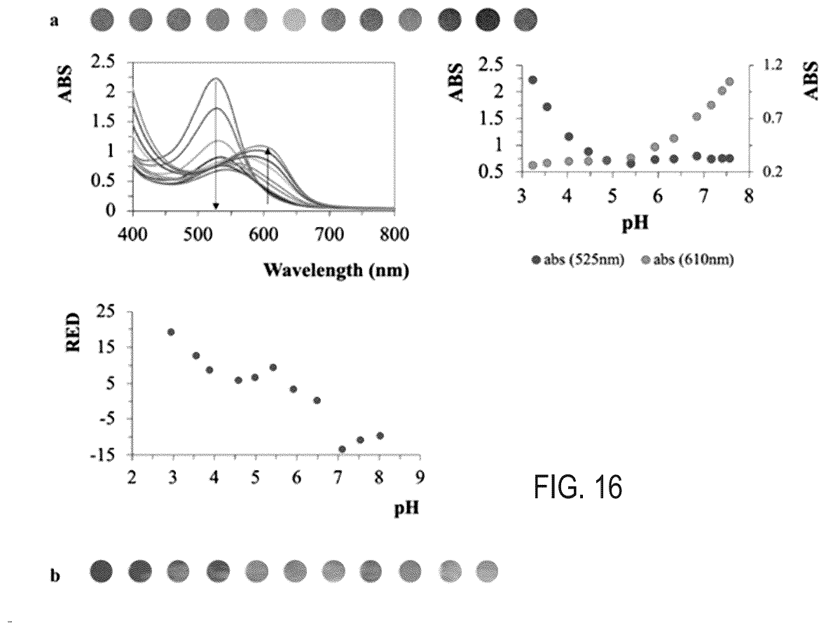

Silk-based colorimetric cocktails were characterized via UV-VIS spectrophotometry in the range 400-800 nm, at steps of 5 nm. NY silk-based cocktails were sensitive within the range pH 5.5-8.5 (FIG. 1). BG/CPR silk-based cocktails were sensitive within the range pH 5.5-7.5 (FIG. 12). FIG. 13 and FIG. 15 show the behavior of silk fibroin cocktails embedding anthocyanins extracted from blueberries and red cabbage, respectively. They display different color maps and different sensing ranges (i.e., BB, pH 3-4.4 and pH 5.3-7.8; RC, pH 3.4-4.7 and pH 6.2-7.5). Anthocyanins extracted from diverse fruits and vegetables slightly differ in the chemical structure causing variations in the sensing range of the overall cocktails.^{[2,3]} BB and RC can be combined together in different ratios to adjust the color maps accounting for different sensing ranges. FIG. 14 and FIG. 16 show the color map and sensing response of BB/RC in volume ratios of 1:2 (i.e., pH 3.3-4 and pH 6.5-7.7) and 2:1 (i.e., pH 3.2-4.5 and pH 5.9-7.6), respectively. The different combinations highlight the opportunity to finely tune the sensing range depending on the performances of the final application.

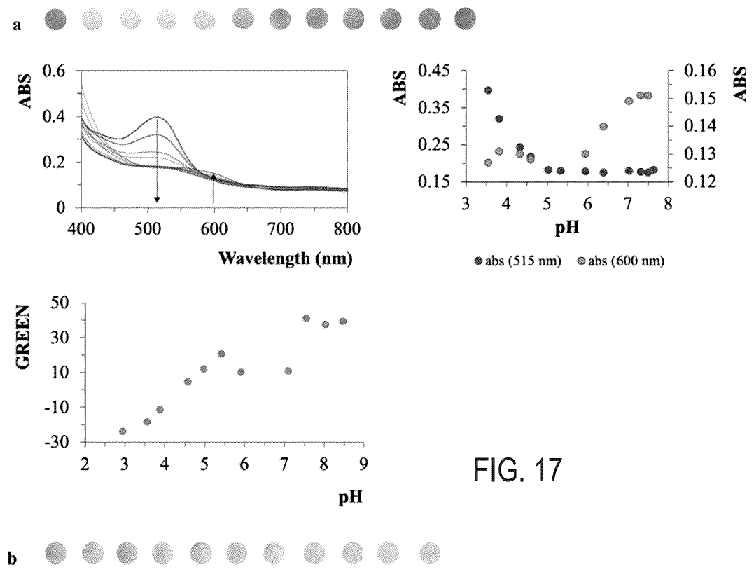

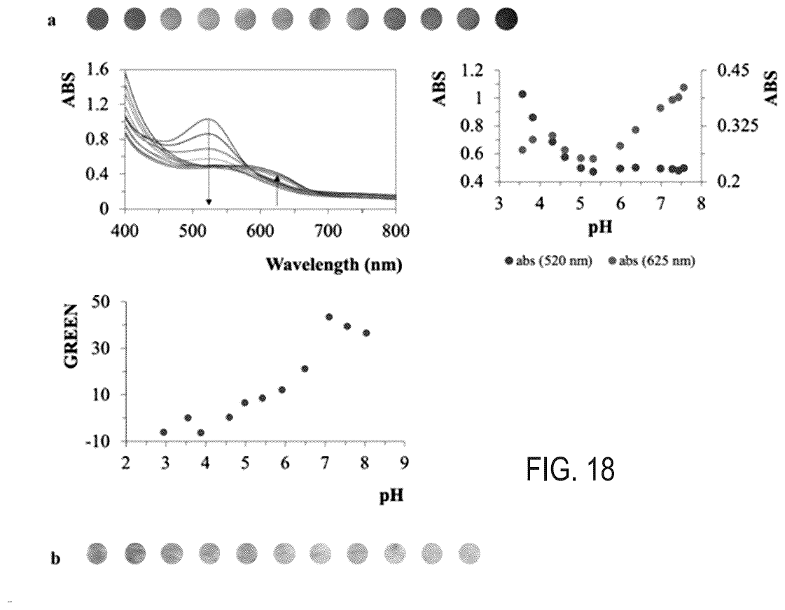

Carotene extracted from nectarine skins can also track pH variations via color changes in real-time. FIG. 17 shows the color maps for carotene extracted from nectarine skins. Color differences were noticeable but the sensitivity of silk-based nectarine mixtures was pretty low (i.e., absorbance peaks with max intensity of 0.4 ABS) and the sensing range (i.e., pH 3.5-4.6) was one unit of pH. Carotene molecules had to be combined with other color sensing molecules such as anthocyanins extracted from blueberries to improve the overall performances and enlarge the sensing range. Performances were slightly improved (FIG. 18) since the sensitivity and sensing range were both extended (i.e., pH 3.6-4.6 and 5.3-7.6) but the results are not as good as those obtained with commercially available pH indicators and anthocyanins employed at higher concentrations. Carotene based mixtures reduced sensitivity and sensing range seemed to be mainly dictated by the low yield obtained using the extraction procedure aforementioned. Performances may be easily improved changing the extraction procedure or concentrating the final extract to achieve increased carotene concentrations that will augment the sensitivity of the biomaterial-based cocktails.

Performances of Colorimetric Mouth Conformable Interfaces

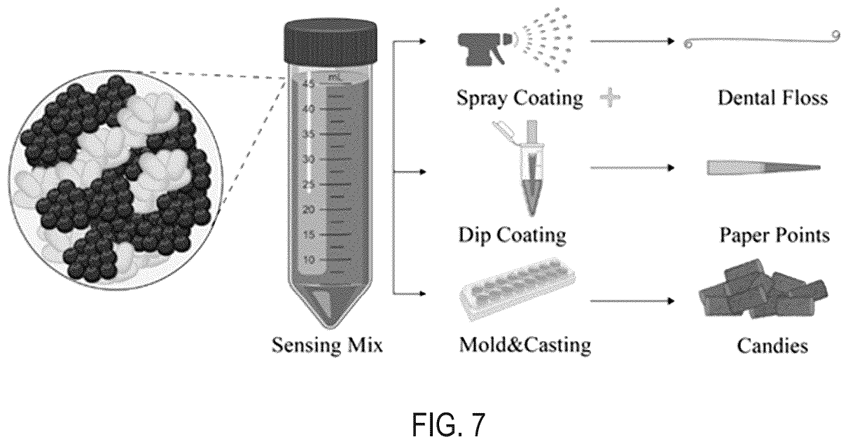

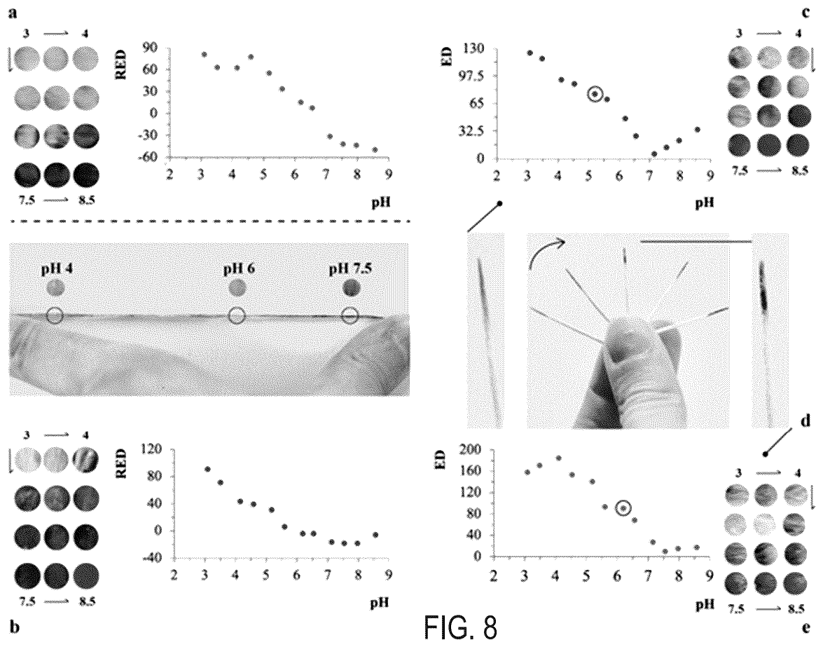

Spray coated dental floss embed commercial pH indicators such as NY (i.e., Sensitivity RED: −40.8±2 within the range pH 4.5-7.5) (FIG. 7, panel a) or a combination of BG/CPR (i.e., Sensitivity RED: −25.9±2 within the range pH 3-7) (FIG. 7, panel b). Highly absorbent paper points were dip coated with commercially available pH indicators such as the combination of BG/CPR (i.e., Sensitivity ED: −28±1.9 within the range pH 3-7) (FIG. 7, panel c) or NY (i.e., Sensitivity ED:−49.2±3.2 within the range pH 4-7.5) (FIG. 7, panel e). Naturally available pH indicators were also used to dip coat paper points. BB points were sensitive within multiple ranges: pH 3.5-5 (Sensitivity Red: −17.4±2.2); pH 6-7 (Sensitivity Red: −21.4±2.9); pH 8-8.5 (Sensitivity Red: −40.8) (FIG. 8, panel a). Paper points realized with a combination of BB/RC (i.e., ratio 1:2 v/v) were sensitive within multiple ranges: pH 3-4 (Sensitivity Green: 34.2±4.8); pH 4.5-5 (Sensitivity Green: 19.7); pH 5.5-6 (Sensitivity Green: 21.4); pH 6.5-7 (Sensitivity Green: 17); pH 7.5-8.5 (Sensitivity Green: 23.1±0.5) (FIG. 8, panel c). Naturally available pH indicators were also embedded within fully edible mixtures that allowed the realization of color changing lollipop like devices. BB based lollipops were sensitive within the range pH 4.5-5 (Sensitivity ED: −17.7±3.5) (FIG. 8, panel d). Nectarine based lollipops were sensitive within multiple ranges: pH 3-4 (Sensitivity Red:−27.1±4.8) and pH 7-8 (Sensitivity Red:−35.5±0.5) (FIG. 8, panel c). The pKₐ of all pH indicators was shifted after embedment on solid substrates. This phenomenon was previously observed elsewhere and it is attributable to the dye being immobilized within a microenvironment that differs from the standard liquid (i.e., silk in this publication) in which the dyes are usually dissolved.

Discussion of the Performance of the Bioresponsive Interfaces

Colorimetric sensing offers a compelling strategy for practical and rapid detection of local pH variations inside the mouth. The examples herein provide an approach based on the use of biomaterial-based mixtures to functionalize inert interfaces of dental appliances/substrates, such as dental floss, endodontic paper points, and dental aligners to diagnose local areas of the oral cavity.

The examples further demonstrate bioresponsive candy-like devices that embed color changing biochemical reporters to continuously transduce pH variations in saliva. The sensing formulations based on naturally derived biomaterials provide the advantage of using water as a solvent and of being processed at room temperature, thus enabling direct integration and stabilization of labile sensing molecules into cocktails based on a liquid suspension of silk fibroin. Such cocktails have the capability to stabilize sensing agents, such as pH indicators or labile pH sensing molecules (e.g., anthocyanin, carotenoid, etc.) extracted from fruits (e.g., blueberries, nectarine skins) and vegetables (e.g., red cabbage). The cocktails can be tuned for spray-coating and dip coating or to create solid bioreactive interfaces with long shelf-life under ordinary (i.e., refrigeration-less) storage conditions without compromising their biochemical reporting functionality.

An exemplary method of producing the oral sampling devices is illustrated in FIG. 6. The method includes combining bioreactive formulations with oral sampling support substrates used in dentistry by either coating or incorporation of reagents onto such substrates, such as dental floss (i.e., through spray coating), onto highly absorbent cellulose paper points used for root canals (i.e., by dip coating), and by developing standalone reactive candies by direct inclusion of fruit-extracted reagents.

The biomaterial-based bioresponsive formulations were first characterized in liquid format via UV-VIS spectrophotometry. Analytical performance was preserved when all the pH indicators were integrated into silk fibroin-based mixtures thus validating the possibility for their use to activate inert substrates of different kinds as described above.

The biomaterial-based formulations were first used to encapsulate sensing agents, such as pH-responsive molecules, namely a combination of bromocresol green (BG)/chlorophenol red (CPR), and nitrazine yellow (NY), which were spray coated on dental floss (FIG. 7, panels a and b). pH variations were monitored in real-time by measuring variations in the intensity of red, green, or blue (RGB) channels or of a combination of the three of them, by measuring the Euclidean Distance (ED) of their colorimetric response. The color channels exhibited varying levels of sensitivity. FIG. 7, panels a and b show variations of Red intensity for both NY and BG/CPR within the pH range 4.5-7.5 and 3-7, respectively (see Supporting Information for details). FIG. 7, panel b shows how different areas of the same dental floss can colorimetrically detect different pH values (i.e., pH 4, 6, and 7.5) allowing for accurate real-time tracking of inter-tooth pH values, which is particularly important to monitor given the difficulty to reach these areas with conventional oral hygiene.

The same sensing agents (e.g., pH indicators) were used to dip coat highly absorbent paper points that are currently employed for standard clinical treatment for root canal (FIG. 7, panels c, d, and e). Color changing paper points may allow localized monitoring of pH variations in hardly accessible areas inside the oral cavity and inside the teeth. FIG. 7, panels c and e show intensity variations of ED for both BG/CPR and NY within the pH range 3-8.5. BG/CPR points are sensitive within the range pH 3-7 and NY points are sensitive within the range pH 4-7.5 (see Supporting Information for details). FIG. 7, panel d shows the colorimetric response of paper points at different pH, with the insets highlighting performance at pH 5 (i.e., for BG/CPR points) and pH 6 (i.e., for NY points).

BG/CPR and NY were selected to functionalize both dental floss and paper points due to their overlapping pKa over the range of interest for non-invasive monitoring of pH in saliva. When using indicators, undesirable leaching might constitute an issue to be addressed. As such, the concentration of the pH indicators used in the devices (e.g., concentration of ˜1 ug/mm2 per coated area). Leaching of pH indicators may be undesirable. Additional strategies can be adopted including the addition of a semi-permeable biocompatible layer that would drive and confine the saliva interaction with the bioactive interface. In the case of the paper points, direct contact of the active interface with the oral cavity can be easily avoided. Due to the high absorbance of the paper, the tip of the point is used to passively draw the saliva into contact with the sensing formulation area that is located farther from the tip.

Another strategy that can be adopted to overcome the drawbacks mentioned above is to rely on naturally available pH indicators such as anthocyanins. These are safe to consume and can be readily extracted from fruits (e.g., blackberries, cherries, blueberries, etc.) and vegetables (e.g., red radishes, red cabbage, red beets, etc.).

For this purpose, extraction and testing of indicators based on palatable fruits (i.e., blueberries, (BB)) and vegetables (i.e., red cabbage (RC)) was performed. FIG. 8, panels a and c show the Red and Green channel intensities variations for BB and a combination of BB/RC, respectively, within the pH range 3-8.5. BB and BB/RC points are sensitive within multiple ranges (see Supporting Information for details). FIG. 8, panels b and d show colorimetric shifts of paper pH points at different pH highlighting variations recorded at pH 6 for both BB and BB/RC.

Both BB and BB/RC are sensitive over the critical pH range of relevance to screen for caries development where they can easily detect changes occurring at pH 5.5, indicative of increased risk for the onset of caries and a critical level at which enamel undergoes demineralization. This strategy would constitute a useful and practical in-home diagnostic tool that allows identification of imbalance between cycles of enamel demineralization and remineralization processes without the need of dental x-rays.

Identification of demineralization in such early stages can be utilized to shift the balance into proper remineralization with an adequate supply of calcium, phosphate and fluoride ions thus providing a method to control the demineralization process and prevent impairment of deeper regions in the enamel layer, where dental caries become irreversible.

Enamel demineralization of occlusal surfaces can be easily detected by visual examination. On the contrary, at present, enamel demineralization occurring between teeth can be only detected by x-rays, converting inter-teeth cavities into harmful players affecting the overall health of the mouth. The use of the functionalized paper points or other oral sampling devices described herein offer a viable, low-cost alternative that can be also employed outside the clinical practice for early diagnostic of hard-to-detect caries and thus intervene promptly to revert demineralization lesions and avoid dentist intervention. This method can also reduce the amount of routinely dental x-rays taken for diagnostics and monitoring purposes.

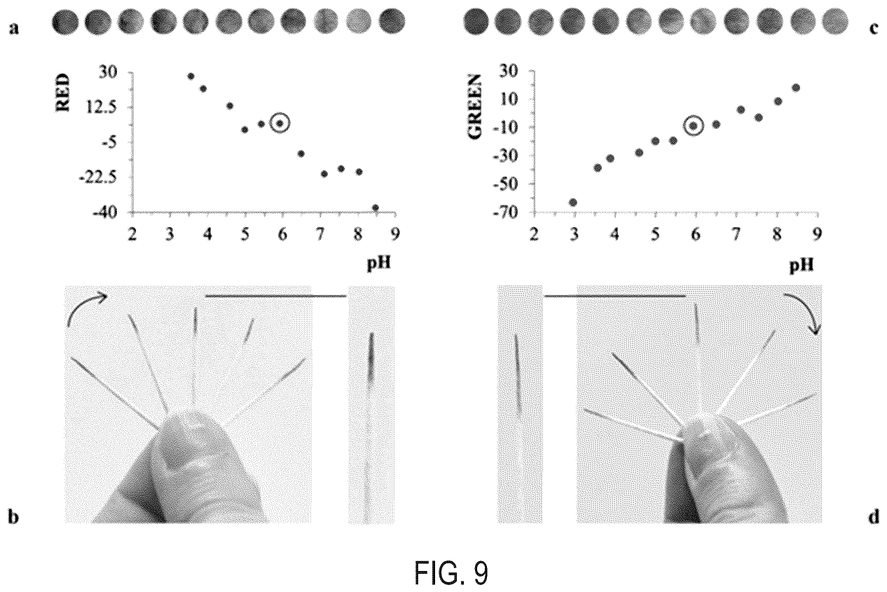

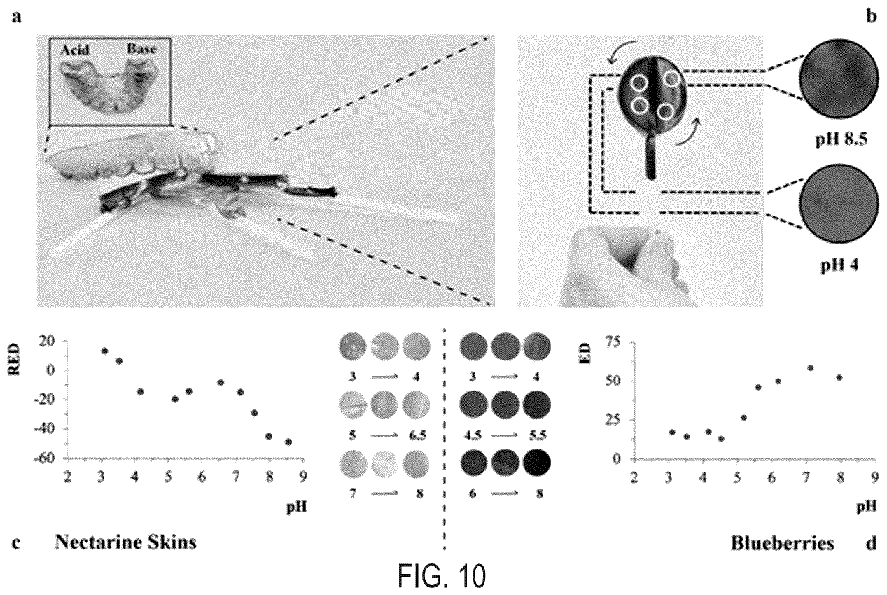

The use of fruit-extracted pH indicators allows for alternate approaches for monitoring overall pH variations in saliva that take advantage of the benign nature of the formulation and enables to redefine sensing formats conveniently for everyday use. To demonstrate the utility of this approach, fully edible colorimetric devices in the format of lollipops were realized (FIG. 9, panel a). These devices embed the naturally available pH indicators into silk fibroin formulations. FIG. 9, panel b shows a lollipop embedding anthocyanins extracted from BB that can be exposed to saliva after introduction inside the oral cavity. The insets show the colorimetric responses recorded at pH 4 and pH 8.5. It is possible to detect variations in the range pH 4.5-7 (i.e., Sensitivity ED: −17.7±3.5), which covers the relevant pH ranges of interest (FIG. 9, panel d) to screen for the salivary buffering capacity and overall oral pH levels. pH sensitive consumable devices with different flavors can be realized changing the type of edible indicator.

FIG. 9, panel c shows the response of bioresponsive lollipops based on carotenoids (i.e., nectarine skin) able to detect pH variations in strongly acidic (i.e., pH 3-4, sensitivity RED: −27.1±4.8) and neutral to mildly basic conditions (i.e., pH 7-8, sensitivity RED:−35.5±0.5). Candy-like devices can be useful in different scenarios where pH variations in whole saliva need to be continuously monitored or in younger demographics. The assessment of children's salivary pH is important when kids are undergoing therapeutic drug intakes that can affect baseline mouth conditions. From a broader medical perspective, acidic environments are usually observed in the oral cavity of cancer patients due to the anaerobic metabolism of glucose in hypoxic conditions created by the tumor acting as a favorable factor for the tumor cells to survive and grow in uncontrolled conditions.

Moreover, radiation therapy impairs the function of the salivary glands which further reduces the oral pH. Monitoring the evolution of patients' salivary pH experiencing cancer treatment accounts for the success of radiotherapy that usually causes a shift in pH (i.e., from initial pretty acidic salivary pH to increased pH values during cancer pathogenesis and radiotherapy sessions). Therefore, functionalizing oral devices such as dental aligners/retainers may be used for prolonged periods of time to allow long term monitoring (FIG. 9, panel a).

pH is just the first of many analytes that can be detected by the devices similar to those here proposed. All naturally available pH indicators proved to be effective in monitoring variations of biological samples within specific ranges with preserved stability over time ensured by integration in functional silk fibroin formulations. Other pH indicators are available for extraction from fruits, vegetables, plants, and flowers paving the way for the implementation of palatable pH sensing devices.

Functionalized mouth conformable interfaces that can non-invasively monitor saliva outside the clinical practice offer new possible paradigms to change the management of oral cavity treatment. A library of assays may be integrated in dental floss, candies, and dental aligners. The devices are able to account in a colorimetric fashion for chemical and physical variations occurring in the mouth. Moreover, multi-biomarkers detection will provide analytical reports establishing eventual correlations that can account for users' health conditions.

The present disclosure has described one or more preferred aspects, and it should be appreciated that many equivalents, alternatives, variations, and modifications, aside from those expressly stated, are possible and within the scope of the invention.

1. An oral sampling device for chemical examination of an oral cavity, the oral sampling device comprising:
an oral sampling support substrate; and
a bioresponsive interface coupled to the oral sampling support substrate, the bioresponsive interface composed of a biopolymeric matrix comprising a sensing agent,
wherein the bioresponsive interface undergoes a color change in response to an environmental parameter in a region of interest in the oral cavity.

2. The oral sampling device of claim 1, wherein the sensing agent is composed of a pH sensing agent.

3. The oral sampling device of claim 2, wherein the pH sensing agent is selected from bromocresol green, chlorophenol red, or nitrazine yellow.

4. The oral sampling device of claim 2, wherein the pH sensing agent is an anthocyanin.

5. The oral sampling device of claim 2, wherein the pH sensing agent is a carotenoid.

6. The oral sampling device of claim 1, wherein the bioresponsive interface or the biopolymeric matrix undergoes a color change in response to a pH change in the oral cavity.

7. The oral sampling device of claim 1, wherein the biopolymeric matrix includes a biopolymer selected from the group consisting of actin, collagen, catenin, claudin, coilin, elastin, elaunin, extensin, fibrillin, fibroin, keratin, lamin, laminin, tublin, zein protein, and combinations thereof.

8. The oral sampling device of claim 1, wherein the biopolymeric matrix comprises silk fibroin.

9. The oral sampling device of claim 1, wherein the bioresponsive interface is coated onto an exterior surface of the oral sampling support substrate.

10. The oral sampling device of claim 1, wherein the bioresponsive interface is intermixed within a body of the oral sampling support substrate.

11. The oral sampling device of claim 1, wherein at least a portion of the oral sampling support substrate is sized to fit between a subject's teeth.

12. The oral sampling device of claim 1, wherein at least a portion of the oral sampling support substrate is configured to wrap around a subject's tooth.

13. The oral sampling device of claim 1, wherein the oral sampling support substrate has a form factor of a dental aligner or a retainer.

14. The oral sampling device of claim 13, wherein the dental aligner has a U-shaped base having an outer labial wall, an inner lingual wall, and an intervening tray floor, and wherein the dental aligner has one or more of the following properties:
(i) wherein the bioresponsive interface is coated on an exterior surface of the outer labial wall, the inner lingual wall, or the intervening tray floor;
(ii) wherein the bioresponsive interface is coated on an interior surface of the outer labial wall, the inner lingual wall, or the intervening tray floor; and
(iii) wherein the bioresponsive interface is intermixed within a body of the outer labial wall, the inner lingual wall, or the intervening tray floor.

15. The oral sampling device of claim 14, wherein the intervening tray floor includes a positive mold of at least one of a subject's teeth.

16. The oral sampling device of claim 1, wherein the oral sampling device has a form factor of a lollipop, wherein the oral sampling support substrate is composed of an elongate stick and the bioresponsive interface has a form of a hardened matrix mounted on the elongate stick.

17. The oral sampling device of claim 1, wherein the oral sampling device has a form factor of dental floss, wherein the oral sampling support substrate is composed an elongate string and the bioresponsive interface is coupled to an exterior surface of the elongate string.

18. The oral sampling device of claim 1, wherein the oral sampling device has a form factor of candy, wherein the oral sampling support substrate is composed of an edible matrix, and the bioresponsive interface is coupled to an exterior surface of the edible matrix or intermixed within the edible matrix.

19. The oral sampling device of claim 1, wherein the oral sampling device has a form factor of a paper point, wherein the oral sampling support substrate is composed of an absorbent material having a cylindrical body and at least one tapered end, wherein the bioresponsive interface is coupled to an exterior surface of the at least one tapered end.

20. (canceled)

21. (canceled)

22. A system for analyzing a bioresponsive interface, the system comprising:
an oral sampling device comprising a bioresponsive interface, wherein the bioresponsive interface undergoes a color change in response to an environmental parameter in a region of interest in an oral cavity;
a memory containing a machine readable medium comprising machine executable code having stored instructions thereon for performing a method of analyzing the bioresponsive interface;
a camera configured to photograph a region of interest on the oral sampling device; and
a control system in electrical communication with the memory, the control system configured to execute the machine executable code to cause the control system to:
determine a pH of the region of interest on the oral sampling device based on a color of the region of interest; and
output a report that includes the pH of the region of interest.

23.-31. (canceled)
